(19) United States
(12) Patent Application Publication
(10) Pub. No.: US 2026/0045369 A1
(43) Pub. Date: Feb. 12, 2026

(54) SYSTEM AND METHOD FOR AGGREGATING INSIGHTS FROM DATASETS DISPERSED ACROSS MULTIPLE DISPARATE PROPRIETARY INFORMATION SYSTEMS

(71) Applicant: Svafy LLC, Chandler, AZ (US)

(72) Inventor: Kumar BALASUBRAMANIAN, Chandler, AZ (US)

(21) Appl. No.: 19/295,481

(22) Filed: Aug. 8, 2025

Related U.S. Application Data
(60) Provisional application No. 63/681,718, filed on Aug. 9, 2024.

(51) Int. Cl. G16H 50/70

(52) U.S. Cl. CPC G16H 50/70

(57) ABSTRACT

A system and method for aggregating insights from disparate proprietary information systems is disclosed. The system includes an insight aggregation server configured to receive a resource data mapper from each target entity of a plurality of target entities, each target entity having a target entity database. The resource data mapper maps its respective database to a virtual information system (VIS). The server is also configured to receive an insight request from a requesting entity in natural-language text form and transform it into a VIS-aligned query using a query interpreter engine, then transform the VIS-aligned query into target entity queries using a query mapper engine and the resource data mappers. Each target entity query is sent to the target entity it is aligned with, verified for authorization, and executed by that target entity database. The execution results from all target entities are then aggregated and provided to the requesting entity.

### RELATED APPLICATIONS

This application claims the benefit of U.S. provisional patent application 63/681,718, filed Aug. 9, 2024 titled “System and Method for Aggregating Insights from Datasets Dispersed Across Multiple Disparate Proprietary Information Systems,” the entirety of the disclosure of which is hereby incorporated by this reference.

### TECHNICAL FIELD

Aspects of this document relate generally to data insights from multiple information systems.

### BACKGROUND

In today's data-driven world, the value of data cannot be overstated. Data constitutes the bedrock for deriving valuable insights, facilitating informed decision-making, and creating avenues for monetization. Businesses and organizations spanning various sectors leverage data to gain competitive advantages, optimize operations, and develop innovative products and services. However, actualizing the full potential of data poses substantial challenges, particularly when data is dispersed across multiple, disparate systems owned by different entities.

One primary challenge is the complexity of forming coherent and effective queries for insights across multiple, disparate proprietary systems. Each data repository often utilizes unique data structures, schemas, and query languages, complicating the process for a single entity to efficiently gather and integrate insightful information from diverse sources. This fragmentation leads to inefficiencies and significant delays in data insight retrieval processes. For instance, within the healthcare industry, an insurance provider seeking comprehensive health data insights about its members must navigate numerous electronic health record (EHR) systems, each with its proprietary data organization and access protocols. EHR vendors may hesitate to invest in providing proprietary interfaces to insurance providers due to the development and maintenance costs as well as proprietary interface adoption barriers, even though such data sharing could enhance healthcare outcomes. This fragmentation impedes timely and accurate data insight aggregation, crucial for effective decision-making and service provision. Furthermore, this limited data sharing reduces the overall value that can be extracted from the data.

Moreover, the sensitive and confidential nature of much of the data introduces another layer of complexity. Sensitive data, such as personal health information, financial records, or proprietary business intelligence, must be handled with utmost care to ensure compliance with privacy laws and regulations, such as HIPAA in the healthcare sector. The need to protect sensitive information while still enabling meaningful data insights presents a challenging balancing act. For instance, healthcare providers must ensure that patient data is securely managed and shared only with authorized entities, complicating the process of data insight aggregation and analysis.

For example, health insurance companies requiring detailed health information about their members must reverse-engineer claims data to identify specific patient encounters with healthcare providers. This process is not only time-consuming but also often incomplete, as claims data typically lacks comprehensive health information. Consequently, insurance companies invest substantial resources in alternative methods, such as home health checkups, to obtain the necessary data. Not only are these fee-based data acquisition methods expensive, they also face the additional challenge of having an uncertain return-on-investment, as there is no assurance that the acquired data will be relevant to the insight being sought.

Another example involves the difficulty in obtaining specific health insights, such as identifying members who have been prescribed a medication or referred a particular procedure by their physician, but there was no follow up action on it. Traditional methods to address this necessitate insurance companies to request broad datasets and sift through the information to extract the desired insights. This process is time-consuming, expensive, prone to errors and omissions, and often fails to provide a complete picture, as the necessary data may be scattered across different EHR systems that do not communicate with each other.

### SUMMARY

According to one aspect, a system for insight aggregation includes an insight aggregation server having a processor and a memory. The processor is configured to receive a resource data mapper from each target entity belonging to a plurality of target entities communicatively coupled to the insight aggregation server through a network. Each target entity has a target entity database, and the resource data mapper received from a target entity is specific to the target entity database of that target entity and maps the target entity database of that target entity to a virtual information system (VIS) that is based upon a technical specification that is a published technical specification. The processor is further configured to receive an insight request from a requesting entity communicatively coupled to the insight aggregation server through the network, the insight request being text in prose form or text derived from a spoken request. The processor is configured to transform the insight request into a VIS-aligned query using a query interpreter engine, the VIS-aligned query being aligned with the virtual information system, and also transform the VIS-aligned query into a plurality of target entity queries using a query mapper engine. The query mapper engine is configured to produce a target entity query aligned with a particular target entity using the resource data mapper provided by that target entity. The processor is configured to send each target entity query of the plurality of target entity queries produced by the query mapper engine to the target entity whose target entity database the target entity query is aligned with, to be executed by that target entity database. The processor is also configured to receive a result, through the network, from each target entity of the plurality of target entities, the result produced in response to the target entity query being executed by the target entity database, aggregate a plurality of results into an aggregated insight, and send the aggregated insight to the requesting entity through the network. The insight request comprises terms missing from the technical specification.

Particular embodiments may comprise one or more of the following features. The technical specification may be a Health Level Seven (HL7) standard. The technical specification may be the Fast Healthcare Interoperability Resources specification. At least one of the query interpreter engine and the query mapper engine may be a transformer-based Large Language Model.

According to another aspect of the disclosure, a system for insight aggregation includes an insight aggregation server having a processor and a memory. The processor is configured to receive a resource data mapper from each target entity belonging to a plurality of target entities communicatively coupled to the insight aggregation server through a network. Each target entity includes a target entity database, and the resource data mapper received from a target entity is specific to the target entity database of that target entity and maps the target entity database of that target entity to a virtual information system (VIS) that is based upon a technical specification. The processor is also configured to receive an insight request from a requesting entity communicatively coupled to the insight aggregation server through the network. The processor is additionally configured to transform the insight request into a VIS-aligned query using a query interpreter engine, the VIS-aligned query being aligned with the virtual information system, and also transform the VIS-aligned query into a plurality of target entity queries using a query mapper engine. The query mapper engine is configured to produce a target entity query aligned with a particular target entity using the resource data mapper provided by that target entity. The processor is configured to send each target entity query of the plurality of target entity queries produced by the query mapper engine to the target entity whose target entity database the target entity query is aligned with, to be executed by that target entity database. The processor is also configured to receive a result, through the network, from each target entity of the plurality of target entities, the result produced in response to the target entity query being executed by the target entity database, aggregate a plurality of results into an aggregated insight, and send the aggregated insight to the requesting entity through the network.

Particular embodiments may comprise one or more of the following features. The insight request may be text in prose form. The technical specification may be a published technical specification. The insight request may include terms missing from the technical specification. The technical specification may be a Health Level Seven (HL7) standard. The technical specification may be the Fast Healthcare Interoperability Resources specification. At least one of the query interpreter engine and the query mapper engine may be a transformer-based Large Language Model. The processor may be further configured to validate the VIS-aligned query before transformation by the query mapper engine.

According to yet another aspect of the disclosure, a method for insight aggregation includes receiving a resource data mapper from each target entity belonging to a plurality of target entities communicatively coupled to an insight aggregation server through a network. Each target entity includes a target entity database, and the resource data mapper received from a target entity is specific to the target entity database of that target entity and maps the target entity database of that target entity to a virtual information system (VIS) that is based upon a technical specification. The method also includes receiving an insight request from a requesting entity communicatively coupled to the insight aggregation server through the network, and transforming the insight request into a VIS-aligned query using a query interpreter engine. The VIS-aligned query is aligned with the virtual information system. For each target entity of the plurality of target entities, the method includes transforming the VIS-aligned query into a target entity query using a query mapper engine, with the query mapper engine configured to produce a target entity query aligned with the target entity using the resource data mapper provided by that target entity. The method also includes providing the target entity query to the target entity database of that target entity for execution, and receiving a result through the network from the target entity, the result produced in response to the target entity query being executed by the target entity database. Finally, the method includes aggregating a plurality of results into an aggregated insight, and sending the aggregated insight to the requesting entity through the network.

Particular embodiments may comprise one or more of the following features. The insight request may be text in prose form. The technical specification may be a published technical specification. The insight request may include terms missing from the technical specification. The technical specification may be a Health Level Seven (HL7) standard. The technical specification may be the Fast Healthcare Interoperability Resources specification. At least one of the query interpreter engine and the query mapper engine may be a transformer-based Large Language Model. The method may also include validating the VIS-aligned query for syntactic and/or semantic correctness before transforming the VIS-aligned query with the query mapper engine.

Unless specifically noted, it is intended that the words and phrases in the specification and the claims be given their plain, ordinary, and accustomed meaning to those of ordinary skill in the applicable arts. The inventors are fully aware that they can be their own lexicographers if desired. The inventors expressly elect, as their own lexicographers, to use only the plain and ordinary meaning of terms in the specification and claims unless they clearly state otherwise and then further, expressly set forth the “special” definition of that term and explain how it differs from the plain and ordinary meaning. Absent such clear statements of intent to apply a “special” definition, it is the inventors' intent and desire that the simple, plain and ordinary meaning to the terms be applied to the interpretation of the specification and claims.

The word “exemplary,” “example,” or various forms thereof are used herein to mean serving as an example, instance, or illustration. Any aspect or design described herein as “exemplary” or as an “example” is not necessarily to be construed as preferred or advantageous over other aspects or designs. Furthermore, examples are provided solely for purposes of clarity and understanding and are not meant to limit or restrict the disclosed subject matter or relevant portions of this disclosure in any manner. It is to be appreciated that a myriad of additional or alternate examples of varying scope could have been presented, but have been omitted for purposes of brevity.

The inventors are also aware of the normal precepts of English grammar. Thus, if a noun, term, or phrase is intended to be further characterized, specified, or narrowed in some way, then such noun, term, or phrase will expressly include additional adjectives, descriptive terms, or other modifiers in accordance with the normal precepts of English grammar. Absent the use of such adjectives, descriptive terms, or modifiers, it is the intent that such nouns, terms, or phrases be given their plain, and ordinary English meaning to those skilled in the applicable arts as set forth above.

Further, the inventors are fully informed of the standards and application of the special provisions of 35 U.S.C. § 112 (f). Thus, the use of the words “function,” “means” or “step” in the Detailed Description or Description of the Drawings or claims is not intended to somehow indicate a desire to invoke the special provisions of 35 U.S.C. § 112 (f), to define the invention. To the contrary, if the provisions of 35 U.S.C. § 112 (f) are sought to be invoked to define the inventions, the claims will specifically and expressly state the exact phrases “means for” or “step for”, and will also recite the word “function” (i.e., will state “means for performing the function of [insert function]”), without also reciting in such phrases any structure, material or act in support of the function. Thus, even when the claims recite a “means for performing the function of . . . ” or “step for performing the function of . . . ,” if the claims also recite any structure, material or acts in support of that means or step, or that perform the recited function, then it is the clear intention of the inventors not to invoke the provisions of 35 U.S.C. § 112 (f). Moreover, even if the provisions of 35 U.S.C. § 112 (f) are invoked to define the claimed aspects, it is intended that these aspects not be limited only to the specific structure, material or acts that are described in the preferred embodiments, but in addition, include any and all structures, materials or acts that perform the claimed function as described in alternative embodiments or forms of the disclosure, or that are well known present or later-developed, equivalent structures, material or acts for performing the claimed function.

The foregoing and other aspects, features, and advantages will be apparent to those artisans of ordinary skill in the art from the DETAILED DESCRIPTION and DRAWINGS, and from the CLAIMS.

### BRIEF DESCRIPTION OF THE DRAWINGS

The disclosure will hereinafter be described in conjunction with the appended drawings, where like designations denote like elements, and:

FIG. 1 is a schematic view of an interoperable insight aggregation system; and

FIG. 2 is a schematic process view of a method for aggregating insights from datasets dispersed across multiple disparate proprietary information systems.

### DETAILED DESCRIPTION

While this disclosure includes a number of embodiments in many different forms, there is shown in the drawings and will herein be described in detail particular embodiments with the understanding that the present disclosure is to be considered as an exemplification of the principles of the disclosed methods and systems, and is not intended to limit the broad aspect of the disclosed concepts to the embodiments illustrated.

In today's data-driven world, the value of data cannot be overstated. Data constitutes the bedrock for deriving valuable insights, facilitating informed decision-making, and creating avenues for monetization. Businesses and organizations spanning various sectors (e.g., healthcare, finance, retail, logistics, manufacturing, etc.) leverage data to gain competitive advantages, optimize operations, and develop innovative products and services tailored to evolving customer demands.

However, actualizing the full potential of data poses substantial challenges, particularly when data is dispersed across multiple, disparate systems owned and controlled by different entities. These systems are often siloed for historical, technical, or legal reasons, and lack the interoperability necessary for seamless integration and aggregation. This fragmentation not only inhibits comprehensive analysis but also creates bottlenecks that can affect everything from operational efficiency to regulatory compliance and strategic planning.

One primary challenge is the complexity of forming coherent and effective queries for insights across multiple, disparate proprietary systems. Each data repository often utilizes unique data structures, schemas, terminologies, and query languages, complicating the process for a single entity to gather and integrate information from diverse sources efficiently. For instance, some systems may rely on SQL-based queries while others use NoSQL or bespoke APIs, each with differing data access models. This fragmentation leads to inefficiencies and significant delays in data retrieval processes, especially when real-time or near-real-time insights are required.

For example, within the healthcare industry, an insurance provider seeking comprehensive health data about its members must navigate numerous electronic health record (EHR) systems, each with its proprietary data organization, access protocols, and compliance requirements. These systems may vary not only in structure but also in the granularity and timeliness of the data they contain. EHR vendors may hesitate to invest in providing proprietary interfaces to insurance providers due to the development and maintenance costs as well as proprietary interface adoption barriers, even though such data sharing could enhance healthcare outcomes, support population health management, reduce duplicative testing, and lower long-term costs. This fragmentation impedes timely and accurate aggregated data insights, which is crucial for effective decision-making, risk stratification, and service provision. Furthermore, this limited data sharing reduces the overall value that can be extracted from the data, especially in areas such as predictive modeling, chronic disease management, and preventive care.

Additionally, a significant tension exists for data holders between the opportunity to monetize data and the investments needed to ensure security, access authorization, and regulatory compliance, in providing interested parties direct access to their proprietary information. Data holders are understandably cautious about exposing their data assets due to concerns regarding data security, loss of competitive advantage, potential liability, and the need to maintain compliance with a growing body of data protection regulations. These concerns are amplified in regulated industries or where data subjects' rights are paramount. This reluctance often results in limited data sharing and collaboration, reinforcing existing silos and further stifling innovation. For example, EHR vendors may hesitate to share detailed patient information with insurance providers due to investments needed to address privacy concerns and regulatory constraints, such as those imposed by HIPAA or state-level privacy laws, even though such data sharing could enhance healthcare outcomes, improve claims processing, and enable more accurate risk assessment and care coordination.

Moreover, the sensitive and confidential nature of much of the data introduces another layer of complexity. Sensitive data (e.g., personal health information, financial records, geolocation data, proprietary business intelligence, etc.) must be handled with utmost care to ensure compliance with privacy laws and regulations, such as HIPAA in the healthcare sector, the Gramm-Leach-Bliley Act in finance, or international frameworks like the GDPR. Mishandling of such data can lead to severe financial penalties, reputational damage, and legal liabilities. The need to protect sensitive information while still enabling meaningful data insights presents a challenging balancing act that requires careful governance, robust access controls, and sometimes complex anonymization or de-identification protocols. For instance, healthcare providers must ensure that patient data is securely managed and shared only with authorized entities, often requiring robust consent management frameworks, audit trails, and encryption at rest and in transit. These requirements complicate the process of data aggregation and analysis, particularly when multiple stakeholders are involved.

For example, health insurance companies requiring detailed health information about their members must often reverse-engineer claims data to identify specific patient encounters with healthcare providers. This process is not only time-consuming but also often incomplete and error-prone, as claims data typically lacks detailed clinical information, including provider notes, lab results, and imaging data. Moreover, claims data may be delayed or aggregated in ways that obscure the chronological sequence or contextual relationships among clinical events. Consequently, insurance companies invest substantial resources in alternative methods, such as home health checkups, to obtain the necessary data. These in-person visits, while valuable, are logistically complex and expensive to scale. They also face the additional challenge of having an uncertain return-on-investment, as there is no assurance that the acquired data will be relevant to the insight being sought or that the data acquired will be comprehensive, particularly when the data collection is not directly aligned with the targeted decision-making goal.

Another example involves the difficulty in obtaining specific health insights, such as identifying members who have been prescribed a medication or referred a particular procedure by their physician, but for whom no follow-up action occurred. Traditional methods to address this necessitate insurance companies to request broad datasets, sometimes from multiple sources, and sift through the information to extract the desired insights. This process is time-consuming, expensive, prone to errors and omissions, and often fails to provide a complete picture, as the necessary data may be scattered across different EHR systems that do not communicate with each other. Even where data is theoretically accessible, the lack of standardized semantics, coding systems, and interoperability frameworks creates practical barriers to integration. As a result, critical gaps in care continuity, treatment adherence, and risk stratification persist despite the availability of advanced analytics tools and methodologies.

Contemplated herein is a system and method for aggregating insights from datasets spread across multiple disparate proprietary information systems. The contemplated interoperable insight aggregation system is able to take a simple text input (e.g., prose) expressing a desired insight and convert it into customized queries bespoke for each target entity's proprietary information system. Reluctant data holders no longer have to provide direct access, or deal with forming the appropriate queries to satisfy a requested data insight. According to various embodiments, the system and method contemplated herein automatically generates these queries specific to each proprietary system, including data authorization checks, and within the secure environments of said systems. The target entity simply runs the provided query, and sends back the result. The structure of the proprietary data system is not shared or exposed, and granting direct access to external entities is not needed, according to various embodiments. This maintains privacy and opens up opportunities for data monetization while requiring minimal effort on the part of the data holder.

Additionally, the system contemplated herein allows a target entity to monetize their data without incurring any of the software development or maintenance costs that are often required when sharing with conventional information systems. According to various embodiments, the contemplated system will allow a target entity to serve multiple requesting entities without needing to change their own system. Similarly, according to various embodiments, the contemplated system will allow a requesting entity to submit its request to multiple target entities without needing to customize its request for each target entity.

In the context of the present description and the claims that follow, an insight is simply a piece of information. In some embodiments, an insight may be a piece of raw information retrieved from an information system. In other embodiments, an insight may represent information that has been distilled, to some degree, from an information system. For example, rather than replicating all patient records from a proprietary information system, an insight might represent a compilation of particular pieces of information (e.g., date of last bloodwork, cholesterol levels, etc.) for records that meet a particular criteria (e.g., a specific individual, all people above a particular age, etc.). Advantageously, requesting an insight rather than raw data or entire records allows a target entity to restrict certain aspects of their data (e.g., PII, billing information, etc.) while still being able to monetize their data. It should be noted that in some embodiments, an insight may also be raw data pulled from an information system without any processing, filtering, or distillation. Put differently, an insight is at least a piece of information taken directly from an information system, but may also be information resulting from an analysis, distillation, summarization, or other similar operation being performed on the data, or a subset of the data, within an information system.

According to various embodiments, the interoperable insight aggregation system is able to quickly and accurately translate an insight request, which may be provided in prose form in some embodiments, into a SQL query bespoke for a proprietary system through the use of two AI-driven engines, the query interpreter engine and the query mapper engine. The query interpreter engine takes the insight request and converts it into an intermediate form. This intermediate form is sent to a query mapper engine, which translates the queries from the intermediate form into the proprietary format of the proprietary system. This query mapper engine can be used to translate any query provided in the intermediate form into a query compatible with the proprietary format of the proprietary system, independent of who the requesting entity is.

The interoperable insight aggregation system allows data to remain within the target entity's secure environments by executing the queries locally and only sending authorized insights back to the source entity, thus protecting the proprietary and sensitive nature of the raw data. In some embodiments, the contemplated system and method ensures that data mapping information provided by target entities stays within their firewalls, and the process only involves the exchange of insights rather than raw data, thereby maintaining the confidentiality and security of sensitive information. Furthermore, the contemplated interoperable insight aggregation system and method automates the entire process of querying, mapping, and aggregating data insights using AI engines, reducing the need for manual data handling and enabling faster, more accurate, and scalable data retrieval and analysis, according to various embodiments.

The following discussion is done in the context of systems using SQL queries. However, it should be noted that the system and method contemplated herein may be adapted for use with other database languages, protocols, models, and/or types. The contemplated system facilitates communication with other data systems that only “speak” in a proprietary language. As will be discussed below, this language gap is bridged through the use of two query engines that speak with each other using a shared language described by an internal, virtual information system (VIS). According to various embodiments, the system and method contemplated herein may be adapted for use with any type of information retrieval system, including those not yet developed. Thus, while the following discussion is done in the context of SQL queries, it is not limited to SQL.

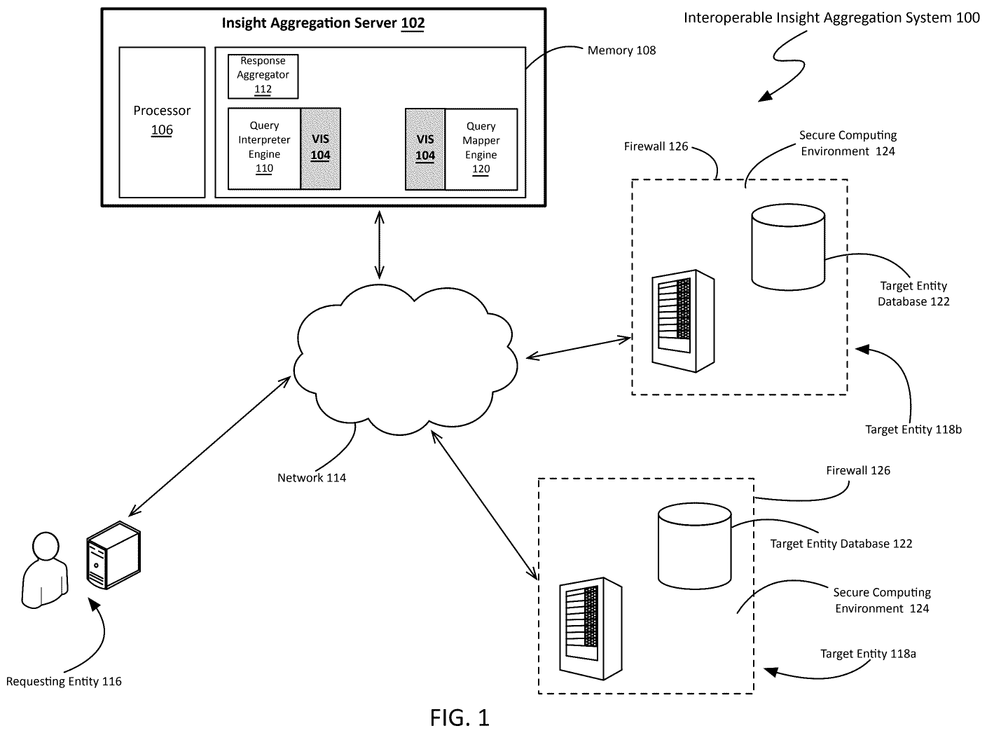

FIG. 1 shows a schematic view of a non-limiting example of an interoperable insight aggregation system 100. According to various embodiments, the interoperable insight aggregation system 100 enables a requesting entity 116 to describe its desired insight using a text input (i.e., an insight request), submit the request simultaneously to multiple approved target entities 118, and seamlessly derive aggregated insights 210 from their responses, without the need to be aware of the disparate internal proprietary information systems of the target entities 118.

As shown, the system 100 comprises an insight aggregation server 102 having a processor 106 and a memory 108. The insight aggregation server 102 serves as a hub that coordinates the question-response interaction between a requesting entity 116 and one or more target entities 118. In the context of the present description and the claims that follow, an entity (i.e., requesting entity 116, target entity 118) is one or more individuals requesting and/or providing data, insights, and the like. An entity may range from a single individual user to a corporation with thousands of employees. The term entity is also used herein to refer to the computing resources that one or more individuals are utilizing in their role of requesting and/or providing data. Examples include servers, clusters, databases, and any other form of computing devices and environments known in the art. In some embodiments, the contemplated system 100 may be interacted with through a web interface, a consequence being that any device that can run a web browser could be considered an entity.

According to various embodiments, the insight aggregation server 102 may be implemented in any computing environment having a processor 106 and a memory 108 with sufficient resources for the scope of the specific use case for which it is intended. As shown, the insight aggregation server 102 is communicatively coupled to a requesting entity 116 and a plurality of authorized target entities 118 through a network 114 (e.g., the Internet).

The interoperable insight aggregation system 100 contemplated herein is built upon four novel computing elements: the virtual information system 104, the query interpreter engine 110, the query mapper engine 120, and the resource data mapper 200. According to various embodiments, the query interpreter engine 110 is capable of turning a text request for a data insight or analysis into a VIS-aligned query 204 whose structure and atomic data elements are defined by the virtual information system 104 (or VIS). The VIS-aligned query 204 is sent to various authorized target entities 118 pertinent to the desired insight. Each of these target entities 118 possesses a query mapper engine 120 that is under their control. According to various embodiments, these query mapper engines 120 are configured to convert the VIS-aligned query 204 into a target entity query 206 that is compatible with the proprietary data system of a specific target entity database 122. This conversion is made possible through a resource data mapper 200 which maps the VIS-aligned query 204 specification to the target entity query 206 specification; it is defined and, in some embodiments, controlled by the target entity 118. The responses resulting from the execution of the target entity queries 206 by the target entities 118 are aggregated and sent to the requesting entity 116. The virtual information system 104, the query interpreter engine 110, the query mapper engine 120, and the resource data mapper 200 will each be discussed in greater detail, below.

In the context of the present description and the claims that follow, a virtual information system 104 (VIS) is an internal construct or conceptual framework used for intermediate processing, bridging two adaptive engines: the query interpreter engine 110 and the query mapper engine 120. Essentially, the VIS 104 provides a shared context which these two engines can use to communicate with each other. The query interpreter engine 110 and query mapper engine 120 will both be discussed in greater detail, below.

This virtual information system 104 is an abstraction. According to various embodiments, the VIS 104 essentially embodies a database schema, but is not itself a database. Put differently, the VIS 104 is more like a language than a mechanism.

According to various embodiments, the VIS 104 is aligned with a technical specification, meaning that a technical specification is the foundation for the “VIS language”. As a consequence, the queries passed between the query interpreter engine 110 and the query mapper engine 120 are grounded in a standardized schema based on industry specifications, according to various embodiments.

In the context of the present description and the claims that follow, the term “aligned” refers to the semantic and structural correspondence between two data schemas or models, such that elements in one can be meaningfully mapped, interpreted, or translated into the context of the other. More precisely, to say that a system, query, or component is “aligned” with a technical specification (e.g., HL7 FHIR) means that it adheres to the conceptual structure and naming conventions of that specification, uses data elements, types, and relationships defined or derived from that specification, and/or is interoperable with or translatable to systems that implement that specification.

In the context of the present description and the claims that follow, “technical specification” refers to a formalized definition that governs various aspects of data operations. It defines the “what” (e.g., data formats, data elements, data structures, etc.) and the “how” (e.g., an API for exchanging data, etc.) for data operations in a specific context (e.g., for a specific industry, using a specific technology, satisfying a specific requirement, performing a specific task, etc.). Put differently, the technical specification explicitly defines and clearly describes a set of resources, as well as how they behave, how they are represented, and how they are utilized.

In the context of the present description and the claims that follow, a “published” technical specification is a technical specification that is, at the very least, publicly available. In some embodiments, a published technical specification is a technical specification that is the result of an effort to standardize data operations within a particular industry, often created through the collaboration and agreement of multiple entities within said industry. However, it should be noted that while “published” may be desirable, it is in no way required for the VIS 104 to facilitate the intermediate processing. According to various embodiments, the VIS 104 is aligned with a technical specification which may or may not be published. The notoriety and origin of the technical specification is of no import to the operation of the VIS 104 itself. In fact, in one embodiment, the VIS 104 is aligned with a technical specification that is entirely proprietary, which is not an impediment to the operation so long as the two engines are “fluent” in this otherwise secret language. Thus, the terms “technical specification” and “published technical specification” may be used interchangeably within the present description.

Much of this disclosure is done in the context of healthcare use cases, for illustrative purposes. In some embodiments, the VIS 104 may align with a published healthcare technical specification such as the Fast Healthcare Interoperability Resources (FHIR) specification. Other healthcare related examples include the Consolidated Clinical Document Architecture (C-CDA), Integrating the Healthcare Enterprise (IHE), OpenEHR, as well as other Health Level Seven (HL7) standards beyond FHIR.

As is known in the art, FHIR facilitates the exchange of healthcare information between different systems, ensuring that patient data is accessible, consistent, and understandable across various platforms. It enables different systems to work together seamlessly by providing a common framework. FHIR specifies RESTful APIs that enable web-based access to health information, and enables the structured representation of clinical data (e.g., patient demographics, observations, medications, diagnostic reports, etc.).

In other embodiments, the VIS 104 may align with other published technical specifications that may be adapted for other industries or implementation environments including, but not limited to, manufacturing (e.g. Open Platform Communications Unified Architecture, etc.), finance (e.g., ISO 20022, Financial Information exchange, etc.), energy (e.g., IEC 61850, Open Automated Demand Response, etc.), and retail (e.g., GS1, etc.). Those skilled in the art will recognize that still other embodiments of the contemplated system and method may comprise a VIS aligned with other published technical specifications, including technical specifications that do not yet exist.

According to various embodiments, the VIS 104 is confined to a finite set of atomic data elements (i.e., the smallest units of data that carry meaning that cannot be divided further without losing significance) and their associated data types. These are defined in the technical specification with which the VIS 104 is aligned. These atomic data elements serve as building blocks and structure, the vocabulary and grammar of the VIS 104 language.

As a specific, non-limiting example, in an embodiment where the VIS 104 is aligned with the FHIR specification, the “Patient Resource” in the “Base Module” has atomic elements such as family name, given name, name prefix, and name suffix, each of which is defined to be of type ‘string’.

According to various embodiments, the finite set of atomic data elements is partitioned into a finite set of categories, such that related atomic data elements are grouped together. Each of these categories has a unique name, in some embodiments. Likewise, each of the atomic data elements within a category has a unique name. In some embodiments, atomic data elements across different categories are allowed to have the same name. In some embodiments, these categories and associations are defined in the published technical specification. In other embodiments, they are defined in the VIS 104 separate from, but still in compliance with, the published technical specification.

As previously discussed, the virtual information system 104 is abstract. the VIS 104 is essentially a schema for a database that is not expected to exist as an actual information system. According to various embodiments, the virtual information system 104 exists within the contemplated system as a plurality of tables that describe the non-existent database. In some embodiments, each category name in the technical specification will be represented as a table. For example, if there exist 157 categories defined in the technical specification, then there will be 157 tables in the “database schema” of the VIS 104. In some embodiments, the published technical specification would have ensured that the different categories have unique names, thereby ensuring that these tables will also have unique names. Within these tables, all the atomic data elements that are grouped together within a category in the technical specification are represented as the different columns within the table that corresponds to that category, according to various embodiments.

In application, these tables are used as a shared lexicon for the query interpreter engine 110 and the query mapper engine 120, allowing them to speak clearly to each other in ways that will not preclude subsequent “translation” into the proprietary languages used by the various target entities 118. Continuing with the language analogy, if the contemplated system were configured to interface with target entities 118 that each speak a different Romance language, VIS might allow the query interpreter engine 110 and the query mapper engine 120 to speak to each other in Latin.

The query interpreter engine 110 is responsible for translating a text-based insight request from plain language or prose into an SQL query that is aligned with the virtual information system 104 (i.e., a VIS-aligned query), according to various embodiments. As an option, in some embodiments the insight request may be provided to the system as a spoken request that is converted into text by one of the requesting entity 116 (e.g., the device used to interact with the system 100, etc.), the insight aggregation server 102, or a third party communicatively coupled to the interoperable insight aggregation system 100. In some embodiments, the query interpreter engine 110 is built on top of natural language processing (NLP) tools used in conjunction with artificial intelligence, such as transformer-based generative Large Language Models (LLM). Exemplary LLMs include, but are not limited to, OpenAI's ChatGPT, Anthropic's Claude, Google's Gemini, Meta's Llama, and the like.

According to various embodiments, the query interpreter engine 110 is trained to process the input text string (i.e., the insight request 302 or a part thereof) by applying in-depth specifics of the desired SQL language constructs, and produce a SQL Query output that represents the health insight described in the input string (i.e., insight request 302). In some embodiments, the input text string is expected to use terminologies tightly aligned with those used in the published specification. For example, in case of the FHIR specification, the term “observation” in the input text string would refer to the ‘observation’ resource in the clinical module of the FHIR specification. However, in other embodiments, the query interpreter engine 110 may be configured to interpret the meaning of the insight request and put that meaning into the context of the technical specification. That way, in the case of the insight request using terms that are missing from the technical specification, the query interpreter engine 110 can suggest an aligned term to the requesting entity 116, or even make the substitution by itself, in some embodiments.

The SQL query output produced by the query interpreter engine 110 (i.e., the VIS-aligned query) is aligned to the virtual database schemas of the VIS 104, as discussed above. If there were to exist a real-world information system with schemas identical to those of the VIS 104 described earlier in this disclosure, then this SQL query output could be submitted directly to that system for seamless, successful execution.

This SQL query output can be aligned to any desired database management system prevalent in the industry such as PostgreSQL, MySQL, SQLite, Oracle DBMS, IBM DB2, Microsoft SQL Server, and the like. The richer the training dataset provided to the query interpreter engine 110, the stronger will be its alignment to the VIS 104 and the published specification terminologies, and higher will be its accuracy in correctly representing the original intended insight being sought. In some embodiments, the query interpreter engine 110 is adaptive and able to learn such that performance will improve over time as it continuously learns from its experiences in processing large volumes of relevant real-world input text strings. In other embodiments, the AI model of the query interpreter engine 110 may be static, unable to learn new tricks after training is complete (i.e., the model weights are frozen).

In some embodiments, the query interpreter engine 110 can be extended to also accept additional relevant information from the requesting entity 116. For example, the requesting entity 116 may provide a list of values for an atomic data element, and desire to seek responses for the requested insight only within the context where the listed atomic data element has one of those listed values. Those skilled in the art will recognize that other filtering mechanisms may be implemented, to the benefit of the requesting entity 116.

The query mapper engine 120 is responsible for interfacing with the proprietary information system of a particular target entity 118, and translating a VIS-aligned query (i.e., a SQL query that is aligned with the VIS 104) into a query aligned with the target entity's information system, hereinafter referred to as the target entity database 122. According to various embodiments, the query mapper engine 120 may utilize transformer-based artificial intelligence, such as an LLM. This enables it to comprehend the entire SQL context in a VIS-aligned query produced by the query interpreter engine 110, and convert the VIS-aligned query into an equivalent SQL query that is customized for the information system of that target entity 118. The query mapper engine 120 is able to perform this conversion using the information provided by the resource data mapper.

The use of the VIS 104 provides a stable, well-defined context against which each target entity 118 may describe their own proprietary information system (i.e., the target entity database 122). This description is the resource data mapper, which allows the query mapper engine 120 to translate the VIS-aligned SQL query produced by the query interpreter engine 110 into multiple different “languages”. The resource data mapper will be discussed in greater detail, below

According to various embodiments, the insight aggregation server 102 may only require one query mapper engine 120, regardless of the number of target entities 118 and their associated resource data mapper information. In some embodiments, multiple query mapper engines 120 may be used in parallel, should the volume of queries be too large for a single query mapper engine 120 to handle with the desired response time. For example, in some embodiments, each potential target database management system may be assigned its own query mapper engine 120.

Like some embodiments of the query interpreter engine 110 discussed above, some embodiments of the query mapper engine 120 are also able to continuously learn and improve in performance over time through experience in processing large volumes of relevant real-world inputs. In other embodiments, the artificial intelligence used by the query mapper engine 120 (e.g., LLM, etc.) may be static (e.g., the model weights are frozen, etc.).

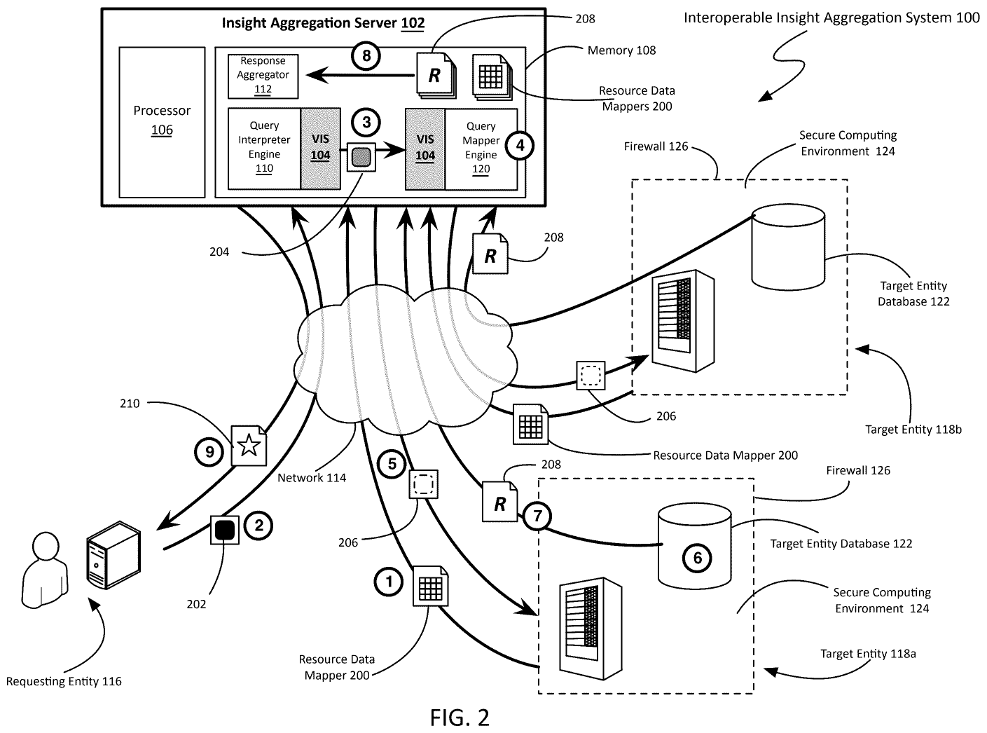

It should be noted that in the non-limiting example of the contemplated interoperable insight aggregation system 100 shown in FIG. 1, the query mapper engine 120 is part of the insight aggregation server 102. In other embodiments, the query mapper engine 120 may be located within the secure computing environment 124 of each target entity 118. This permits a greater degree of privacy for the target entities 118, who would not need to reveal anything about their proprietary systems. Instead, the resource data mapper they define for their system, and the query mapper engine 120, would reside within their firewall 126, receiving VIS-aligned queries, translating them into the form native to the proprietary system, and executing the query. The results are returned to the insight aggregation server 102, just like the embodiments shown in FIGS. 1 and 2.

However, this increased privacy comes at a cost. In embodiments where the query mapper engine 120 is located within the secure computing environment 124 of a target entity 118, the target entity 118 would bear the computational burden of running the query mapper engine 120 which, depending upon the volume of requests and the sophistication of the AI powering the query mapper engine 120, may be significant. In most cases, embodiments where the query mapper engine 120 is local to the insight aggregation server 102 would be preferred.

In the context of the present description and the claims that follow, a resource data mapper is a mapping that correlates the VIS 104 virtual database schema with the proprietary information system of a target entity 118. It can be used by the query mapper engine 120 as a dictionary, allowing the query mapper engine 120 to translate a query formed for execution within the theoretical database of the VIS 104 into a query that will be understood by the proprietary target entity database 122 of a particular target entity 118.

More specifically, the resource data mapper specifies the correlation between the tables and columns of the VIS 104 (theoretical) database and their equivalent tables and columns in the target entity 118's proprietary information system. This information can be conceptualized as a series of rows, where each row would have six elements. Element 1 would specify a VIS table name and element 2 would specify a VIS column name within the VIS 104 table name given in element 1 of this row. Element 3 specifies the datatype of that element in VIS. Together, elements 1 and 2 would specify a unique atomic data element in the published technical specification, and element 3 specifies its datatype. Element 4 would specify a table name and element 5 would specify a column name in the target entity 118's proprietary information system (i.e., target entity database 122), such that the elements 4 and 5 will collectively specify the same atomic data element in the published technical specification that is represented collectively by elements 1 and 2 of this row. Element 6 specifies the datatype of the element in the proprietary information system. According to various embodiments, elements 3 and 6 (i.e., the datatypes) would need to match for the two represented atomic data elements to be considered equivalent.

In some embodiments, and for any combination of elements 1 and 2, if the target entity 118 does not have an equivalent representation in its proprietary information system, then elements 4, 5, and 6 of that row will each be captured as ‘NA’ (indicative of no equivalent information available). In other embodiments, the target entity 118 may only capture the atomic elements that are represented in its proprietary information system. This eliminates the use of ‘NA’ elements.

It should be noted that for all atomic data elements of the published technical specification captured in the target entity 118's proprietary information system, the captured values will need to be in compliance with the published technical specification for that respective atomic data element. In some embodiments, entries in the resource data mapper information will be considered to be case-insensitive.

It should be noted that this conceptualization of the resource data mapper as a table of six element rows is for illustrative purposes, and is not intended to be limiting. Those skilled in the art will recognize that the information embodied in these six elements could be represented and/or organized in other ways, with the same result.

According to various embodiments, the correlation information contained within a resource data mapper could be provided in any of a number of different forms commonly used in information retrieval technologies including, but not limited to, JSON objects, csv files, Python objects like dictionaries or lists, and the like.

Each target entity 118 provides a resource data mapper that describes their proprietary information system. With this information, the query mapper engine 120 is able to transform the VIS-aligned query into a query that can be sent to the target entity 118, who can then execute the target entity query within their controlled and secure computing environment 124 (e.g., behind their firewall 126), yielding the information sought by the requesting entity 116 without needing to provide any form of access to their proprietary information system, nor change their system to conform with the requesting entity 116 or the insight aggregation server 102 protocols or procedures. This makes the contemplated interoperable insight aggregation system 100 easy to adopt, lowering the barrier to entry for the target entities 118 having data to monetize.

FIG. 2 shows a schematic process view of a non-limiting example of a method for aggregating insights from datasets dispersed across multiple disparate proprietary information systems (i.e., target entity databases 122) using an interoperable insight aggregation system 100. The core of this interoperable insight aggregation system 100 is the insight aggregation server 102.

First, a resource data mapper 200 is received from each target entity 118. See ‘circle 1’. Each participating target entity 118 has a proprietary information system that contains data that will be needed to form an aggregated insight 210. The resource data mapper 200 will be used by the insight aggregation server 102 (specifically, the query mapper engine 120) to translate a request for that insight into a query that is compatible with the proprietary information system (i.e., target entity database 122). As previously discussed, each participating entity will provide a resource data mapper 200 specific to their target entity database 122 that defines how their target entity database 122 (i.e. proprietary information system) can best be mapped onto the theoretical database embodied by the virtual information system 104 of the insight aggregation server 102 and described by the associated technical specification. In some embodiments, the interoperable insight aggregation system 100 may comprise an onboarding tool to assist a target entity 118 in the creation of a resource data mapper 200.

It should be noted that while this does not have to be the first step, the insight aggregation server 102 will not be able to form customized target entity queries 206 until it has the information contained in the resource data mapper 200. The insight aggregation server 102 could first receive an insight request 202 from a requesting entity 116, then determine that it does not have a resource data mapper 200 for an indicated target entity 118, or the resource data mapper 200 is outdated, and thus stop the process until that resource data mapper 200 has been obtained or updated.

One of the advantages the contemplated system and method has over conventional methods of sharing information is that through the use of resource data mappers 200, each target entity 118 can keep their proprietary information system safely confined within a secure computing environment 124 behind their firewall 126 without providing any level of access to the requesting entity 116, yet still be able to share authorized insights from their data without requiring them to change their proprietary information system or their internal procedures to participate. This greatly lowers the barrier to entry for organizations wishing to monetize their data without disrupting their normal operations or weakening their security.

A target entity 118 (e.g., first target entity 118a of FIG. 2) may submit a resource data mapper 200 translating the architecture of their target entity database 122 to the specifications of the VIS 104 provided by the insight aggregation server 102 or the party operating said server 102. As a specific, non-limiting example that will be continued throughout this discussion of the contemplated method, that resource data mapper 200 may look like the following:

Resource Data Mapper 200 from First Target Entity 118a

| V1 Table Name | V1 Column Name | V1 Data Type | V2 Table Name | V2 Column Name | V2 Data Type |
|---|---|---|---|---|---|
| encounters | member_id | INT | visits | patient_number | INT |
| encounters | provider_id | INT | visits | practitioner_number | INT |
| encounters | encounter_date | DATE | visits | visit_date | DATE |
| encounters | encounter_id | INT | visits | visit_key | INT |
| encounters | insurer_id | VARCHAR(10) | visits | insurer_id | VARCHAR(10) |
| diagnoses | dx_code | VARCHAR(10) | condition_records | condition_code | VARCHAR(10) |
| diagnoses | encounter_id | INT | condition_records | visit_key | INT |

As shown, the “visits” table of the target entity database 122 maps onto the “encounters” table of the VIS 104. Even though the columns have different names, the data types are the same. It should be noted that in this simple example, one table maps onto another. In other embodiments, the mapping could link columns of a single table with multiple other tables.

Next, the insight aggregation server 102 receives an insight request 202 from a requesting entity 116 that is communicatively coupled to the insight aggregation server 102 through a network 114. See ‘circle 2’. According to various embodiments, an insight request 202 describes the desired insight. In some embodiments, the insight request 202 may also describe the desired output parameters. In some embodiments, the insight request 202 may be a simple text input, using terms that are consistent with, or closely aligned to, the terminologies used in the published technical specification that is the basis for the virtual information system 104.

Continuing with the specific, non-limiting example discussed above, a requesting entity 116 may submit an insight request 202 that states, in natural language, “List members who have been identified with diabetes condition in the last 30 days. Give me Member ID, Provider ID, Encounter Date.” as an insight request 202. In some embodiments, the insight request 202 may also contain additional targeting information. For example, in some embodiments, the requesting entity 116 would also provide a list of approved target entities 118 to whom requests can be presented. This information may be provided in the insight request 202, or at some other point (e.g., a target entity 118 whitelist to be applied to all of the requesting entity 116's insight requests 202, etc.). Similarly, each target entity 118 would provide a list of approved source entities from whom requests can be accepted for processing. According to various embodiments, an entity may be included on the target list for an insight request 202 from a requesting entity 116 if the requesting entity 116 has the target entity 118 in its list of approved target entities 118, and the target entity 118 has the requesting entity 116 in its list of approved source entities.

After receiving the insight request 202, the insight aggregation server 102 transforms the insight request 202 into a VIS-aligned query 204 using the query interpreter engine 110. See ‘circle 3’. Here, the provided insight request 202 is processed using artificial intelligence by the query interpreter engine 110 to produce an intermediate SQL query (i.e., the VIS-aligned query 204) for the virtual information system 104 that is aligned to the VIS 104 and, therefore, aligned with the technical specification that is the basis for the VIS 104, as previously discussed.

The use of the published technical specification as the foundation for the virtual information system 104 is advantageous, as it gives a well-defined and often industry-approved blueprint for the target entities 118 to map their proprietary information systems using the resource data mappers 200. In application, any technical specification could be used here, so long as it is applied consistently within the query interpreter engine 110 and query mapper engine 120 as well as by the target entities 118 when providing resource data mappers 200. The use of a published technical specification makes it easier to achieve that consistency.

Continuing with the specific, non-limiting example discussed above, the query interpreter engine 130 receives the insight request 202 and transforms it into a VIS-aligned query 204:

VIS-Aligned Query 204 of Query Interpreter Engine 110

```
SELECT
  e.member_id as person_id,
  e.provider_id as practitioner_id,
  e.encounter_date as visit_date
FROM encounters e
JOIN diagnoses d
  ON d.encounter_id = e.encounter_id
WHERE
  e.insurer_id = ‘INS-12345’
  AND (d.dx_code LIKE ‘E10%’ OR d.dx_code
  LIKE ‘E11%’)
  AND e.encounter_date >= CURRENT_DATE −
  INTERVAL 30 DAY;
```

Next, the VIS-aligned query 204 is transformed by the insight aggregation server 102 into a number of equivalent target entity queries 206 using the query mapper engine 120. See ‘circle 4’. The query mapper engine 120 is configured to produce an equivalent customized target entity query 206 aligned with a particular target entity 118 using the resource data mapper 200 provided by that target entity 118. This is done using artificial intelligence, according to various embodiments.

Continuing with the specific, non-limiting example from above, the query mapper engine 120 uses the provided resource data mapper 200 to “translate” the VIS-aligned query 204 into one compatible with the target entity database 122 of the first target entity 118a:

Target Entity Query 206 from Query Mapper Engine 120
(aligned to proprietary DB schema of First Target Entity 118a)

```
SELECT
  v.patient_number as person_id,
  v.practitioner_number as practitioner_id,
  v.visit_date as visit_date
FROM visits v
JOIN condition_records c
  ON c.visit_key = v.visit_key
WHERE
  v.insurer_id = ‘INS-12345’
  AND (c.condition_code LIKE ‘E10%’ OR
  c.condition_code LIKE ‘E11%’)
  AND v.visit_date >= CURRENT_DATE −
  INTERVAL 30 DAY;
```

The use of the query mapper engine 120 and the resource data mappers 200 enables a target entity 118 to receive requests for insights from multiple approved requesting entities in a form that can be directly executed on its respective internal proprietary information system, without the need to develop and maintain any custom technologies for each requesting entity 116, nor providing access to the proprietary information system to any requesting entity 116.

In some embodiments, cases where the query mapper engine 120 detects that the VIS-aligned query 204 cannot be successfully transformed and executed by a target entity 118 based on that target entity's resource data mapper 200 information, that query will not be sent to the target entity 118. The query mapper engine 120 may remove that target entity 118 from the target entity 118 list for that query and update the requesting entity 116 with this information.

As the equivalent customized target entity queries 206 are created by the query mapper engine 120, they are sent by the insight aggregation server 102 to their respective target entities 118. See ‘circle 5’. Once the target entity query 206 is received, the target entity 118 can execute that query within their proprietary information system (i.e., target entity database 122). See ‘circle 6’. The results 208 of that execution can then be returned to the insight aggregation server 102 by the target entity 118. See ‘circle 7’.

Continuing with the specific, non-limiting example from above, the first target entity 118a responds to the target entity query 204 by sending the following result 208 to the insight aggregation server 102:

Result 208 from First Target Entity 118a

| person_id | practitioner_id | visit_date |
|---|---|---|
| 882134 | 45017 | 2025 Jul. 14 |

According to various embodiments, the way target entity queries 206 are handled by the target entity 118 may be entirely up to the target entity 118. For instance, they may wish to handle each query 206 in a manual fashion, evaluating what is being requested and if they are comfortable with sharing. However, it is also possible for the target entity 118 to automate some, or all, of this process. As a specific example, in one embodiment, the target entity 118 may establish a workflow that, upon receipt of a target entity query 206, the target entity query 206 is automatically executed and the results 208 of that execution are then automatically returned to the insight aggregation server 102. As an option, various validations may be performed to ensure the automation isn't opening up a weakness in the target entity's security.

In some embodiments, the query mapper engine 120 may validate the customized target entity queries 206 to ensure that the request is only accessing information which the requesting entity 116 is authorized to receive. In some embodiments, these type of access privileges may be defined on a per-entity basis by a target entity 118, alongside the resource data mapper 200. In other embodiments, an authorization check may be embedded within the VIS-aligned query 204 itself, allowing it to be incorporated into the query logic (e.g., an SQL-based target entity query 206, etc.) that the target entity 118 receives.

The insight aggregation server 102 receives the execution results 208 from each of the participating target entities 118, and then aggregates these results 208 into an aggregated insight 210 using a response aggregator 112. See ‘circle 8’. The collective responses of the target entities 118 yield the desired aggregated analytics for the requesting entity 116. According to various embodiments, this is done using a response aggregator 112, which may be configured to produce a final comprehensive response in a format previously specified by the requesting entity 116 (e.g., within the insight request 202, as part of an onboarding process, etc.). This aggregated insight 210 may then be sent to the requesting entity 116 through the network 114. See ‘circle 9’.

Continuing with the specific example, the aggregated insight 210 obtained from the first and second target entities (118a and 118b) in response to the insight request 202 states:

Aggregated Insight 210 (across all relevant target entities):

| person_id | practitioner_id | visit_date |
|---|---|---|
| 882134 | 45017 | 2025 Jul. 14 |
| 775902 | 66183 | 2025 Jul. 21 |

As seen, the information returned by the first target entity 118a regarding the member with MemberID 882134 has been combined with information received from another target entity (e.g., second target entity 118b, etc.), resulting in this aggregated insight 210. That other information was obtained in response to a target entity query 206 that was created using a different resource data mapper 200 and sent to a different target entity 118 (e.g., second target entity 118b, etc.).

Thus, at the end of the overall process, the requesting entity 116, which started with a text input of its desired insight, successfully derives aggregated insights 210 from multiple target entities 118, even though each target entity 118 processed the request on its respective proprietary internal information system (i.e., target entity database 122).

Access authorization plays a crucial role in any information exchange between entities. For example, in the healthcare industry context, it is critical to ensure that all health information accesses are HIPAA compliant. According to various embodiments, access authorization and authentication may be incorporated into the interaction between the requesting entity 116 and the insight aggregation server 102, as well as between the insight aggregation server 102 and the target entities 118 on a per query basis. That is, in some embodiments, access authorization checks may be performed on every query to ensure that any given query has the authorization to access the information it is requesting. For example, the requesting source's identifier may be embedded as part of the query and thereby consistently ensure authorized access, according to various embodiments.

Additionally, various embodiments of the contemplated insight aggregation server 102 may include various validation at various stages. In some embodiments, the system 100 may validate the VIS-aligned query 204 for syntactic and semantic correctness before proceeding with a transformation by the query mapper engine 120. As a specific example, validation techniques may be applied to verify that the VIS-aligned SQL query (i.e., the VIS-aligned query 204) generated by the query interpreter engine 110 indeed matches the insight requested in the text input received from the requesting entity 116. As another example, validation techniques may be applied to verify that the customized SQL query (i.e. the target entity query 206) generated by the query mapper engine 120 is the equivalent of the SQL query generated by the query interpreter engine 110.

This contemplated interoperable insight aggregation system 100 presents a novel automated system that enables a requesting entity 116 to describe its desired insight using a text input, submit the request simultaneously to multiple approved target entities 118, and seamlessly derive aggregated insights 210 from their responses, without the need to be aware of the disparate internal proprietary information systems of the target entities 118. The analytics desired by the requesting entity 116 is executed at each of the designated target entities 118, and only the resulting authorized insights are aggregated at the requesting entity 116, without the need for any raw data to be aggregated by the requesting entity 116. The requesting entity 116 benefits from timely aggregated business insights despite not having access to the underlying raw data which is distributed across many target entities 118. These insights are available to the requesting entity 116 without requiring them to develop and maintain any custom technologies for each target entities 118. The contemplated interoperable insight aggregation system 100 also benefits the target entities 118 by enabling them to achieve better data monetization without the need to develop and maintain any custom technologies for each requesting entity 116. Because the contemplated system does not require participating entities to alter their own proprietary information systems or change the way they use them, nor require conformance to any proprietary APIs of a requesting entity, there is a very low barrier to broad scale adoption and usage across many industries.

It will be understood that implementations are not limited to the specific components disclosed herein, as virtually any components consistent with the intended operation of a system and method for aggregating insights from datasets across multiple disparate proprietary information systems may be utilized. Accordingly, for example, although particular systems, methods, and/or devices for query interpretation, query mapping, and response aggregating may be disclosed, such components may comprise any shape, size, style, type, model, version, class, grade, measurement, concentration, material, weight, quantity, and/or the like consistent with the intended operation of a system and method for aggregating insights from datasets across multiple disparate proprietary information systems may be used. In places where the description above refers to particular implementations of a system and method for aggregating insights from datasets across multiple disparate proprietary information systems it should be readily apparent that a number of modifications may be made without departing from the spirit thereof and that these implementations may be applied to other interoperable information retrieval or database systems.

What is claimed is:

1. A system for insight aggregation, comprising:
an insight aggregation server comprising a processor and a memory, the processor configured to:
receive a resource data mapper from each target entity belonging to a plurality of target entities communicatively coupled to the insight aggregation server through a network, wherein each target entity comprises a target entity database, and wherein the resource data mapper received from a target entity is specific to the target entity database of that target entity and maps the target entity database of that target entity to a virtual information system (VIS) that is based upon a technical specification that is a published technical specification;
receive an insight request from a requesting entity communicatively coupled to the insight aggregation server through the network, the insight request being one of text in prose form and text derived from a spoken request;
transform the insight request into a VIS-aligned query using a query interpreter engine, the VIS-aligned query being aligned with the virtual information system;
transform the VIS-aligned query into a plurality of target entity queries using a query mapper engine, the query mapper engine configured to produce a target entity query aligned with a particular target entity using the resource data mapper provided by that target entity;
send each target entity query of the plurality of target entity queries produced by the query mapper engine to the target entity whose target entity database the target entity query is aligned with, to be authenticated and executed by that target entity database;
receive a result, through the network, from each target entity of the plurality of target entities, the result produced in response to the target entity query being executed by the target entity database;
aggregate a plurality of results into an aggregated insight; and
send the aggregated insight to the requesting entity through the network;
wherein the insight request comprises terms missing from the technical specification.

2. The system of claim 1, wherein the technical specification is a Health Level Seven (HL7) standard.

3. The system of claim 2, wherein the technical specification is the Fast Healthcare Interoperability Resources specification.

4. The system of claim 1, wherein at least one of the query interpreter engine and the query mapper engine is a transformer-based Large Language Model.

5. A system for insight aggregation, comprising:
an insight aggregation server comprising a processor and a memory, the processor configured to:
receive a resource data mapper from each target entity belonging to a plurality of target entities communicatively coupled to the insight aggregation server through a network, wherein each target entity comprises a target entity database, and wherein the resource data mapper received from a target entity is specific to the target entity database of that target entity and maps the target entity database of that target entity to a virtual information system (VIS) that is based upon a technical specification;
receive an insight request from a requesting entity communicatively coupled to the insight aggregation server through the network;
transform the insight request into a VIS-aligned query using a query interpreter engine, the VIS-aligned query being aligned with the virtual information system;
transform the VIS-aligned query into a plurality of target entity queries using a query mapper engine, the query mapper engine configured to produce a target entity query aligned with a particular target entity using the resource data mapper provided by that target entity;
send each target entity query of the plurality of target entity queries produced by the query mapper engine to the target entity whose target entity database the target entity query is aligned with, to be executed by that target entity database;
receive a result, through the network, from each target entity of the plurality of target entities, the result produced in response to the target entity query being executed by the target entity database;
aggregate a plurality of results into an aggregated insight; and
send the aggregated insight to the requesting entity through the network.

6. The system of claim 5, wherein the insight request is text in prose form.

7. The system of claim 5, wherein the technical specification is a published technical specification.

8. The system of claim 5, wherein the insight request comprises terms missing from the technical specification.

9. The system of claim 5, wherein the technical specification is a Health Level Seven (HL7) standard.

10. The system of claim 9, wherein the technical specification is the Fast Healthcare Interoperability Resources specification.

11. The system of claim 5, wherein at least one of the query interpreter engine and the query mapper engine is a transformer-based Large Language Model.

12. The system of claim 5, wherein the processor is further configured to validate the VIS-aligned query before transformation by the query mapper engine.

13. A method for insight aggregation, comprising:
receiving a resource data mapper from each target entity belonging to a plurality of target entities communicatively coupled to an insight aggregation server through a network, wherein each target entity comprises a target entity database, and wherein the resource data mapper received from a target entity is specific to the target entity database of that target entity and maps the target entity database of that target entity to a virtual information system (VIS) that is based upon a technical specification;
receiving an insight request from a requesting entity communicatively coupled to the insight aggregation server through the network;
transforming the insight request into a VIS-aligned query using a query interpreter engine, the VIS-aligned query being aligned with the virtual information system;
for each target entity of the plurality of target entities:
transforming the VIS-aligned query into a target entity query using a query mapper engine, the query mapper engine configured to produce a target entity query aligned with the target entity using the resource data mapper provided by that target entity;
providing the target entity query to the target entity database of that target entity for execution; and
receiving a result through the network from the target entity, the result produced in response to the target entity query being executed by the target entity database;
aggregating a plurality of results into an aggregated insight; and
sending the aggregated insight to the requesting entity through the network.

14. The method of claim 13, wherein the insight request is text in prose form.

15. The method of claim 13, wherein the technical specification is a published technical specification.

16. The method of claim 13, wherein the insight request comprises terms missing from the technical specification.

17. The method of claim 13, wherein the technical specification is a Health Level Seven (HL7) standard.

18. The method of claim 17, wherein the technical specification is the Fast Healthcare Interoperability Resources specification.

19. The method of claim 13, wherein at least one of the query interpreter engine and the query mapper engine is a transformer-based Large Language Model.

20. The method of claim 13, further comprising validating the VIS-aligned query for syntactic and semantic correctness before transforming the VIS-aligned query with the query mapper engine.
